(19) United States
(12) Patent Application Publication
(10) Pub. No.: US 2016/0147083 A1
(43) Pub. Date: May 26, 2016

(54) Focal Length Adjustment

(71) Applicant: ADLENS LIMITED, Oxford (GB)

(72) Inventors: Robert Stevens, Oxford (GB); Benjamin Holland, Oxford (GB); Jon Nisper, Oxford (GB); Graeme Mackenzie, Oxford (GB)

(21) Appl. No.: 14/898,084

(22) PCT Filed: Jun. 13, 2014
(86) PCT No.: PCT/GB2014/051837

§ 371 Date: Dec. 11, 2015

(30) Foreign Application Priority Data
Jun. 14, 2013 (GB) 1310658.8

(51) Int. Cl. G02C 7/08; A61B 3/11; A61B 3/00; G02C 11/04; A61B 3/028; A61B 3/14; G02C 11/00

(52) U.S. Cl. CPC G02C 7/083; A61B 3/14; A61B 3/111; G02C 11/10; G02C 11/04; A61B 3/028; A61B 3/0025

(57) ABSTRACT

A pair of spectacles comprises a pair of variable focal length lenses; an image acquisition system adapted to acquire images of each of a user's eyes; and a controller adapted to analyze the images to monitor the degree of vergence of the user's eyes, and to adjust a focal length of the variable focal length lenses to a value derived directly from the monitored degree of vergence.

This invention relates to a pair of spectacles comprising a pair of variable focal length lenses and to a methods of controlling and calibrating a pair of variable focal lengths lenses in a pair of such spectacles,

Spectacles with variable focal length lenses are useful, for example, to deal with presbyopia. This is a condition that begins to affect people at the onset of middle age in which the eye exhibits a diminished ability to focus on near objects. The condition is progressive and results in many people requiring vision correction to facilitate reading as they age. To complicate matters, the vision correction required is likely to deteriorate with age, resulting in an individual having to replace their spectacles several times as they age. The condition is common in individuals who have otherwise excellent vision, requiring no correction for distance vision. The additional spherical power that is required for a presbyopic individual will depend on their age and also on the working distance. For example, a book is typically held closer than the viewing distance for a computer monitor and might require a different degree of additional spherical power to form a clear image for a presbyopic individual.

Presbyopia is often compounded by myopia or other vision defects, requiring different prescriptions for distance and close-range vision. For example, a myopic individual may require a spherical power of minus 5 dioptres for general vision with an additional plus 2 dioptres to cater for close tasks such as reading. The additional plus 2 dioptres compensates for the eye's inability to focus for reading. Slightly less additional spherical power might be required to enable the individual to watch television or work with a computer monitor comfortably. Variable focal length lenses can be used to deal with presbyopia by allowing the user to change the focal length when they are concentrating on close tasks.

Spectacles having variable focal length lenses also have an adjustment mechanism to enable the user to adjust the focal length of the lenses as they require. However, having to make this adjustment is inconvenient to the user and can be difficult to make accurately if the mechanism is particularly sensitive or has unstable positions along the travel of the mechanism, from which the adjustment can drift or jump.

Attempts have been made to provide automatic adjustment of the focal length of lenses in spectacles. Many of these attempts have relied, at least in part, on rangefinder autofocus systems, which are complicated and can clash with the biological system controlling the vergence of a user's eyes, which is linked to the accommodation of the lens in the eye. This clash tends to occur with users having moderate presbyopia because the rangefinder system will overestimate the degree of correction that is actually required for such users. Needless to say, this will cause discomfort to the user.

Other attempts have been based on detection of the change in polarisation of light from a user's retina, which can be used to detect perfect focus of an image. However, this is rather a complicated approach and is difficult to achieve reliably without taking extreme measures to ensure that the light can always enter the user's pupil and that the reflected light from the retina can be detected.

In addition to these problems, there are also complications in calibrating an automatic system to an individual wearing the spectacles. Naturally, such calibration must be carried out for each individual since the degree of presbyopia will vary amongst individuals and no two will be exactly alike. The calibration of existing rangefinder and polarisation-based system is not straightforward, and is certainly not amenable for use by an optician and certainly not by a user himself.

In accordance with a first aspect of the invention, there is provided a pair of spectacles comprising a pair of variable focal length lenses; an image acquisition system adapted to acquire images of each of a user's eyes; and a controller adapted to analyse the images to monitor the degree of vergence of the user's eyes, and to adjust a focal length of the variable focal length lenses to a value derived directly from the monitored degree of vergence.

In accordance with a second aspect of the invention, there is provided a method of controlling the focal length of a pair of variable focal length lenses in a pair of spectacles, the method comprising acquiring images of each of a user's eyes; analysing the images to monitor the degree of vergence of the user's eyes; and adjusting a focal length of the variable focal length lenses to a value derived directly from the monitored degree of vergence.

Hence, the invention provides a way of automatically adjusting the focal length of lenses in spectacles, relying only on the degree of vergence without requiring any calculation of the distance from the eyes or lenses to an object being viewed by a user. Vergence is the simultaneous movement of the eyes in opposite directions to obtain or maintain single binocular vision, the eyes moving together when looking at a nearby object and apart when looking at a distant object. The accommodation of the lens in the eye is linked to the degree of vergence. The invention makes use of this phenomenon to determine the appropriate focal length depending only on the vergence. There is no need for complicated rangefinding or polarisation detection systems, and a much more reliable system is obtained as a result. The above-mentioned problems are therefore overcome by the invention.

Where we refer to “variable focal length lenses” in this specification, it should be understood that this encompasses lenses where only a region of the lens has a variable focal length as well as lenses where the entire lens has a variable focal length. Reference to varying the focal length of a lens should be understood to encompass variation of the focal length in the variable focal length regions of such lenses,

It is important to note that at no point is a distance to the object (for example, from the user's eyes or the lenses) calculated or measured or used in any way by the invention. Typically, the only factor that must be monitored is the vergence and this is used directly to derive the required value for the focal length. However, in some embodiments, other secondary factors, such as the ambient light level, may be used to make minor adjustments to the focal length.

The acquired images may be of the whole of each of the user's eyes or of only a part of each of the user's eyes. Typically, the acquired images will include corresponding parts of each of the user's eyes.

In the case where the acquired images include corresponding parts of each of the user's eyes, the controller may be adapted to analyse the images to monitor the degree of vergence of the user's eyes by monitoring the distance between the corresponding parts. Thus, the method may comprise, in the calibration and/or operational mode, analysing the images to monitor the degree of vergence of the user's eyes by monitoring the distance between the corresponding parts. The distance between the corresponding parts varies directly with the degree of vergence and can therefore be used to provide a measure of the vergence of the user's eyes.

The corresponding parts of each of the user's eyes may be any parts of the eyes that are readily detectable so that their relative locations and hence separation can be monitored. For example, the corresponding parts of the user's eyes may be on the limbus, which is the boundary between the sclera and the cornea. Since there is typically a high contrast between the sclera (which is white) and the cornea, the limbus is relatively straightforward to detect using standard image processing techniques, such as thresholding and edge detection to locate the limbus. Typically in this case, the corresponding points will be at the same degree of rotation from a reference radial axis extending outwards from the centroid of the limbus, the locations of which can be calculated after the limbus has been detected as described above.

However, in a preferred embodiment the image acquisition system is adapted to acquire images including at least part of both of a user's pupils, and the controller is adapted to analyse the images to monitor the interpupillary distance. Thus, in this preferred embodiment the corresponding parts of the user's eyes are parts of the user's pupils.

Similarly, the method may, in accordance with this preferred embodiment, comprise acquiring images including at least part of both of a user's pupils, and analysing the images to monitor the interpupillary distance.

Thus, in this preferred embodiment, the corresponding parts of the user's eyes are on the pupils, the distance between the two parts representing the interpupillary distance. The pupil is relatively straightforward to detect since there is typically a high contrast between it and the surrounding iris so that standard image processing techniques, such as thresholding and edge detection, can be used to locate the edge of the pupil. The corresponding parts could then be the centroids of the pupils, the locations of which can be calculated after the edge of the pupil has been detected.

Typically, the image acquisition system comprises one or more cameras for acquiring images including the corresponding parts of each of a user's eyes.

Where more than one camera is used, the image acquisition system may process the images from each camera to produce a single composite image made up from the images acquired by each camera. Standard image stitching techniques can be used for this purpose. Where a single composite image is to be produced in this way, the cameras should preferably have overlapping fields of view to enable an image stitching algorithm to work.

In other embodiments, multiple cameras may be used (typically one for each eye) where the fields of view do not overlap. Each camera would be initially calibrated during manufacturing. This is necessary to account for different nominal parameters, such as frame size, base curve, Pantoscopic and Dihedral angles, and nominal prescriptive interpupillary distance. During an initial user calibration process, a fixed reference point in each field of view would then be found. This can be done by scanning successive captured images from each camera for a respective immobile point (which is detectable because it will not move between successive captured images), such as a tear duct. The distance between the cameras (which is known from the frame geometry) and between each camera and the respective immobile point (which can be measured using image processing techniques) can then be used to determine the separation between the two immobile points even though they do not appear in the same image. By measuring the separation between each immobile point and the corresponding parts of the user's eyes (e.g. the pupils), the distance between the corresponding parts of the user's eyes can be monitored.

In a particularly preferred embodiment, the image acquisition system comprises one or more light sources arranged to illuminate the user's eyes, thereby to form one or more Purkinje images in each of the user's eyes, corresponding Purkinje images in each of the user's eyes defining the corresponding parts of each of the user's eyes.

The method may therefore further comprise illuminating the user's eyes to form one or more Purkinje images in each of the user's eyes, corresponding Purkinje images in each of the user's eyes defining the corresponding parts of each of the user's eyes.

Purkinje images are reflections (in this case of the one or more lights sources) from the structure of the eye. Since there may be reflections from more than one part of the structure of the eye, it is possible for there to be more than one Purkinje image formed. Typically, it is possible to form up to four Purkinje images. The first Purkinje image is the most intense and is the reflection from the outer surface of the cornea. The corresponding Purkinje images mentioned above are therefore usually the first Purkinje images formed in the user's eyes as a result of illumination by the one or more light sources. Typically, the first Purkinje image will be formed on the cornea over the pupil. Since it is such an intense image, it is easily detectable against the dark background of the pupil.

The controller is typically adapted to analyse the images to monitor the distance between the corresponding parts of each of the user's eyes by detecting the corresponding Purkinje images, calculating the location of the centroid of each of the corresponding Purkinje images, and calculating the distance between the centroids.

Thus, the method may further comprise analysing the images to monitor the distance between the corresponding parts of each of the user's eyes by detecting the corresponding Purkinje images, calculating the location of the centroid of each of the corresponding Purkinje images, and calculating the distance between the centroids.

The distance will usually be calculated as a number of pixels in an image.

The controller is normally further adapted to adjust the focal lengths to be adjusted by retrieving an actuation control signal level from a look-up table, and applying the actuation control signal level to one or more actuators coupled to the variable focal length lenses for adjusting their focal lengths. This provides a straightforward way to cause the correct actuation of the variable focal length lenses to be made depending only on the distance between corresponding points on the user's eyes.

The method therefore normally further comprises causing the focal lengths to be adjusted by retrieving an actuation control signal level from a look-up table, and applying the actuation control signal level to one or more actuators coupled to the variable focal length lenses for adjusting their focal lengths.

Alternatively, the controller may be further adapted to adjust the focal lengths by calculating an actuation control signal level from an equation relating the degree of vergence of the user's eyes to the focal length of the variable focal length lenses, and applying the actuation control signal level to one or more actuators coupled to the variable focal length lenses for adjusting their focal lengths.

In this alternative, the method may further comprise adjusting the focal lengths by calculating an actuation control signal level from an equation relating the degree of vergence of the user's eyes to the focal length of the variable focal length lenses, and applying the actuation control signal level to one or more actuators coupled to the variable focal length lenses for adjusting their focal lengths.

As can be seen, the value of focal length can therefore be derived directly from the monitored degree of vergence in a variety of ways, including by retrieval from a look-up table or by calculation. The look-up table or calculation may return a value for the actuation control signal level itself or may return the focal length, from which the actuation control signal level is then calculated.

The look-up table may be any data structure that can be indexed or addressed by one variable (in this case, the monitored degree of vergence) to return a value (in this case for the actuation control signal level).

The controller is preferably located entirely on or within a frame housing the pair of variable focal length lenses.

In a preferred embodiment, the controller is switchable into a calibration mode, in which the controller is further adapted to acquire images of each of a user's eyes, adjust the focal length of the variable focal length lenses to each of at least two set points in succession, receive user input at each of the set points to allow a user to indicate when looking at a predetermined object, analyse the images acquired by the image acquisition system to monitor the degree of vergence of the user's eyes in response to receipt of the user input, and generate an equation relating the degree of vergence of the user's eyes to the focal length of the variable focal length lenses from the focal length and the monitored degree of vergence at each set point.

Thus, in accordance with a third aspect of the invention, there is provided a method of calibrating a pair of variable focal length lenses in a pair of spectacles according to this preferred embodiment, the method comprising switching to the calibration mode; acquiring images of each of a user's eyes; adjusting the focal length of the variable focal length lenses to each of at least two set points in succession; receiving user input at each of the set points to allow a user to indicate when looking at a predetermined object; analysing the images to monitor the degree of vergence of the user's eyes in response to receipt of the user input; and generating an equation relating the degree of vergence of the user's eyes to the focal length of the variable focal length lenses from the focal length and the monitored degree of vergence at each set point.

Thus, the invention provides the capability for variable focal length spectacles to be calibrated quite straightforwardly. All that is required is for the degree of vergence exhibited by a user to be captured at at least two set points of focal length. This enables an equation relating these two quantities to be generated, which serves as the calibration on which subsequent operation of the spectacles can be based.

The controller may be adapted to adjust the focal length of the variable focal length lenses to at least one of the set points in response to user input, whereby each set point represents the focal length at which the user perceives the predetermined object to be in focus.

Thus, in the method of the third aspect, the focal length of the variable focal length lenses may be adjusted to at least one of the set points in response to user input, whereby each set point represents the focal length at which the user perceives the predetermined object to be in focus.

The controller may be adapted to adjust the focal length of the variable focal length lenses automatically to a set point associated with infinity focus.

Thus, in the method of the third aspect, the focal length of the variable focal length lenses may be adjusted automatically to a set point associated with infinity focus.

Preferably, the user input that allows the user to indicate when looking at a predetermined object additionally allows the user to indicate that the predetermined object is perceived to be in focus by the user.

There may be only two set points. However, in other embodiments, there may be three or more set points.

The equation may be a linear equation.

The controller is preferably further adapted when in the calibration mode to use the equation to populate a look-up table linking the monitored degree of vergence or distance of the user's eyes to an actuation control signal level for causing one or more actuators to adjust the variable focal length lenses.

Thus, the method according to the third aspect may further comprise using the equation to populate a look-up table linking the monitored degree of vergence or distance exhibited by the user to an actuation control signal level for causing one or more actuators to adjust the variable focal length lenses.

Alternatively, the controller is further adapted to store one or more parameters representing the equation.

In this case, the method according to the third aspect further comprises storing one or more parameters representing the equation

An embodiment of the invention will now be described with reference to the accompanying drawings, in which:

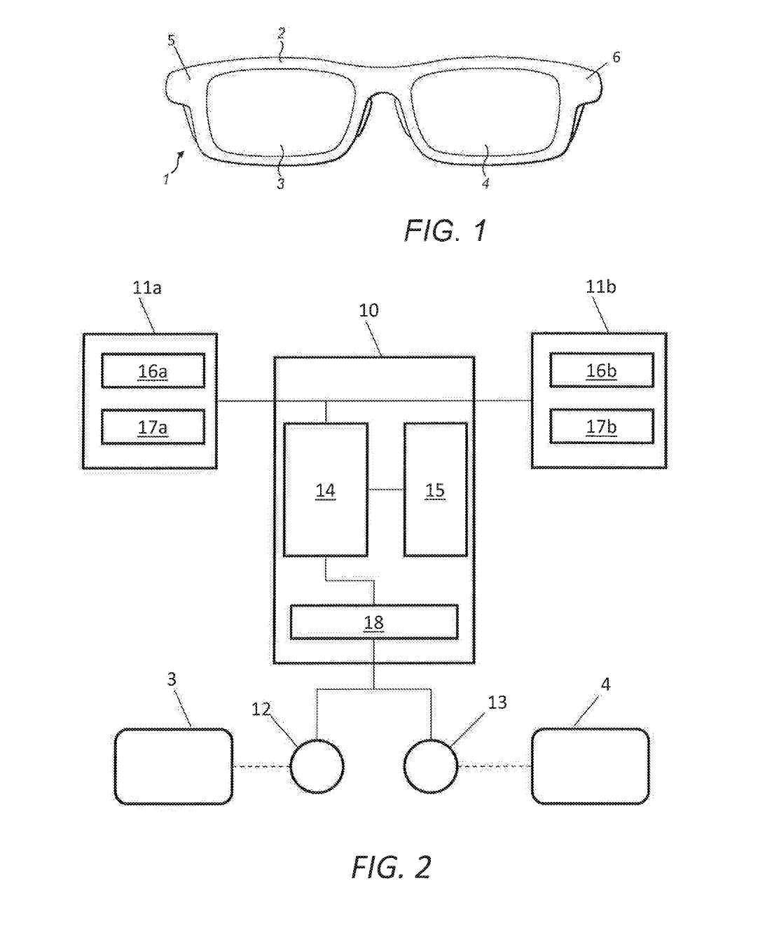

FIG. 1 shows a pair of spectacles according to one embodiment of the invention;

FIG. 2 shows a block diagram of a system embedded in the spectacles of FIG. 1 for carrying out a method according to the invention;

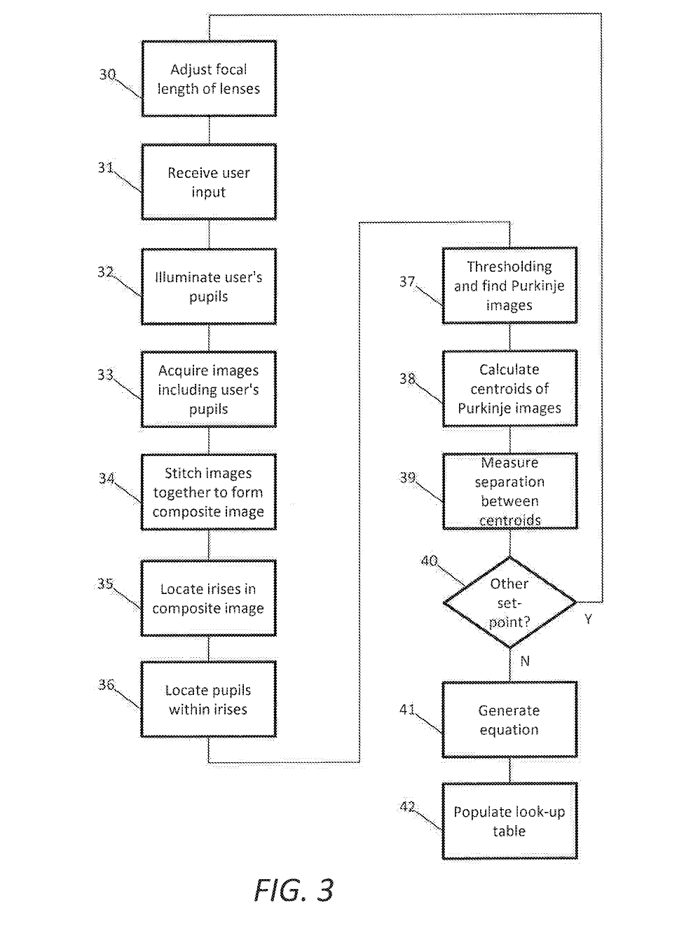

FIG. 3 shows a flowchart of a method for calibrating the spectacles shown in FIG. 1; and

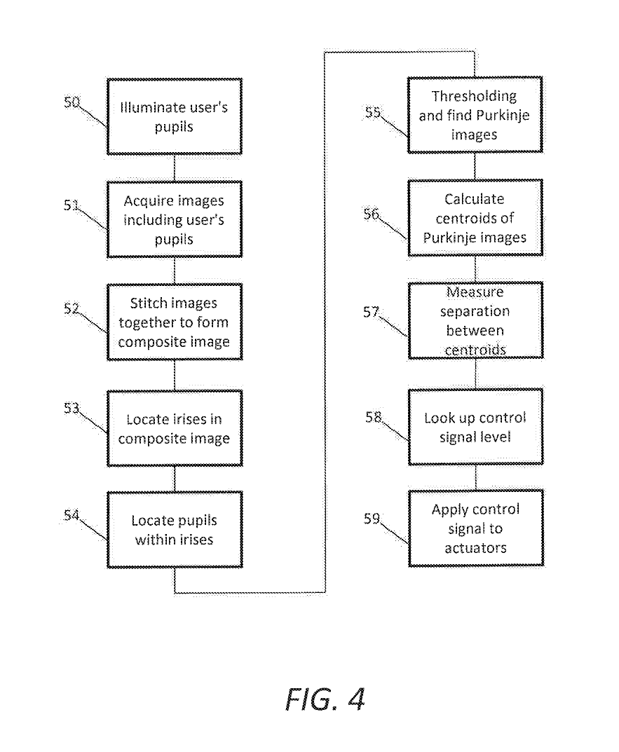

FIG. 4 shows a flowchart of a method for operating the spectacles shown in FIG. 1.

FIG. 1 shows a pair of spectacles 1. The spectacles 1 comprise a frame 2, which houses a pair of lenses 3 and 4. The lenses 3 and 4 are variable focal length lenses. They each comprise a liquid-filled cavity, the anterior surface of which is formed of a flexible membrane. The volume of the cavity or the volume of fluid in the cavity can be adjusted by electrically-operated actuators housed in the temples 5 and 6 of the spectacles 1. As a result of such adjustment, the curvature of the flexible membrane varies, which causes the focal length of the lenses 3 and 4 to vary in sympathy. A detailed description of this type of lens is not included here because it is not necessary to fully understand the invention. Our co-pending application PCT/GB2012/051426 provides a full description of this sort of lens. Other types of variable focal length lenses could be used.

Aside from the electrically-operated actuators, temples 5 and 6 each house parts of an image acquisition system and one of the temples 5 and 6 houses a controller for controlling the operation of the image acquisition system and the electrically-operated actuators. The operation of the controller, image acquisition system and electrically-operated actuators is explained below with reference to FIG. 2.

FIG. 2 shows a block diagram of the system for calibrating the spectacles and subsequently controlling the focal length of the variable focal length lenses 3 and 4. The system comprises a controller 10 coupled to an image acquisition system arranged in two parts 11a and 11b, and to a pair of electrically-operated actuators 12 and 13. Part 11a of the image acquisition system and actuator 12 are associated with lens 3 and housed in temple 5, whereas part 11b of the image acquisition system and actuator 13 are associated with lens 4 and housed in temple 6. The controller 10 may be housed in either of the temples 5 or 6 depending on the design. It is coupled to the parts 11a and 11b of the image acquisition system and actuators 12 and 13 by fine wires running through the frame 2 as necessary. The actuators 12 and 13 may be linear actuators, but in this embodiment are miniature stepper motors mechanically coupled to the lenses 3 and 4 so that rotary motion of the stepper motors causes a corresponding adjustment of the focal length of the lenses 3 and 4.

Controller 10 comprises a microprocessor 14 coupled to a memory 15. The memory 15 stores computer program code for carrying out the methods shown in FIGS. 3 and 4 as will be described below. The microprocessor 14 is also coupled to parts 11a and 11b of the image acquisition system. Each part 11a and 11b of the image acquisition system comprises a respective light source 16a and 16b and a camera 17a and 17b. The light sources 16a and 16b illuminate the user's eyes and the camera captures images of the illuminated eyes for analysis by software running on microprocessor 14 as will be described below. In response to the analysis, the microprocessor 14 provides an output signal to a stepper motor driver 18 to which it is coupled. The stepper motor driver 18 generates pulses to drive the stepper motor actuators 12 and 13 appropriately for adjusting the focal length of lenses 3 and 4 to a value that corresponds to the output signal from the microprocessor 14.

The system of FIG. 2 will also comprise an interface, in the form of one or more buttons on the frame 2 or a wired or wireless interface with an external computer. The interface can be used to switch the controller to a calibration mode and to carry out a calibration process when in the calibration mode. In the event that a wired or wireless interface is provided, this may be any of a variety of interfaces used to couple computing devices, such as USB, a wired network such as Ethernet, a wireless network such as Wi-Fi, Bluetooth or similar.

FIG. 3 shows a flowchart of the method performed by the system (and in particular by microprocessor 14 when executing the program code stored in memory 15) shown in FIG. 2 when switched into the calibration mode. During this process, the user will be asked to look at a series of objects (for example, two objects) placed at various distances from him. For example, the user might be asked to look at a book held at a reading distance, a computer monitor at a typical working distance and an object such as a car or building in the distance. Whilst the user looks at each of the objects in turn, the interpupillary distance will be measured, thereby linking the interpupillary distance (and hence vergence) exhibited by the user to the distance of each of the objects.

The method starts at 30 by adjustment of the focal length of the lenses to a first set point. The first set point of the focal length is that required to enable the user to focus on a predetermined object at a first distance from the user's eyes. The first distance will typically be a distance near to the user, for example the user may be asked to hold a book at a reading distance. The appropriate degree of refractive power (i.e. focal length) that is required for the predetermined object to appear in focus to the user can be determined in a variety of ways. For example, in this embodiment the user controls the focal length of the lenses at 30 with the interface mentioned above and then confirms, again using the interface, at 31 when they are looking at the first object and that the first object appears in focus.

The confirmation received as user input at 31 prompts the system to measure the vergence exhibited by the user's eyes, which of course corresponds to the vergence that will be exhibited whenever the user looks at an object at the same distance as the first object. The measurement of vergence commences in step 32 by illuminating the user's eyes. This is done by the light sources 16a and 16b. These could be permanently powered, although it is preferable if they are illuminated under control of the microprocessor 14 to conserve power when not necessary, for example when in the operational mode and the user has taken off the spectacles 1. As mentioned above, the light sources 16a and 16b are located in the temples 5 and 6. They are directed towards the centre of the user's pupils, and the mounting arrangements holding them in the frame 2 may include an adjustment mechanism to allow the direction of the light emanating from the light sources 16a and 16b to be fine-tuned so that it impinges on the user's corneas directly over the pupils. This ensures that the first Purkinje image is formed over the pupil, where it is readily detectable due to the high contrast between the high intensity Purkinje image and the dark pupil. By ensuring that the light from the light sources 16a and 16b impinges on the anterior surface of the user's eyes at an oblique angle, the first Purkinje image can be formed without the light passing through the pupil and onto the retina. Thus, the user can be relatively unaware of the light from the light sources 16a and 16b so that they do not become a nuisance.

Images of the user's eyes including the sclera, irises and pupils and Purkinje images formed by illumination from light sources 16a and 16b are then acquired at 33. The image acquisition is performed by cameras 17a and 17b, which are located alongside the light sources 16a and 16b in temples 5 and 6.

The image data acquired by cameras 17a and 17b is passed to microprocessor 14, which stitches the image data together at 34 to form a single composite image comprising image data including data representing the user's sclera, irises, pupils and the first Purkinje images formed in the corneas over them. Any standard image stitching algorithm can be used for this purpose.

Next, the image processing performed by microprocessor 14 locates each of the irises in the composite image in step 35. This is typically done using Daugman's algorithm. The pupils are then located within the irises at 36. This is straightforwardly achieved using an edge detection algorithm to locate the boundary within the image data between pixels representing the irises and those representing the pupils. The relatively high contrast between the irises and pupils means that edge detection algorithms can be expected to work well for this task.

A thresholding algorithm is then carried out at 37 on the pixels of image data that represent the pupils. The thresholding algorithm replaces all pixels below a threshold brightness value with black and all those at or above the threshold brightness value with white. The locations of the first Purkinje images are then easily found as a distinct white region within the pupils. This can be achieved using another edge detection process on the thresholded image data that represent the pupils.

The centroids of the first Purkinje images found at 37 as a result of the thresholding operation are calculated at 38. This provides two points representing the geometric centres of the two-dimensional regions defined by the first Purkinje images. The distance between these two points is thus a representation of the interpupillary distance. As the user's eyes move together (a vergence motion caused by looking at a nearer object) the interpupillary distance and thus the distance between the first Purkinje images will decrease. Conversely, as the user's eyes move apart (a vergence motion caused by looking at a more distant object) the interpupillary distance and thus the distance between the first Purkinje images will increase. The number of pixels between the two centroids is measured at 39, this being used to represent the interpupillary distance and hence the vergence exhibited by the user.

The system has now determined the focal length required by the user to perceive the first object as being in focus and the vergence exhibited by the user when looking at an object at the same distance as the first object. At 40, the microprocessor determines whether the vergence exhibited by the user should be measured at another set point for the focal length. A minimum of two must be used, and in practice this is sufficient, although more set points may be used if desired. Thus, in this embodiment two set points are used. The focal length is therefore adjusted again for the second set point at 30. The focal length at the second set point is that required to enable the user to focus on a second predetermined object at a second distance from the user's eyes. The second distance will typically be a distance far from the user, for example the user may be asked to look at a distant car or building at which no additional accommodation would usually be required to be provided by the lenses in the user's eyes. In this case, the focal length may be adjusted either by the user until the second object is perceived to be in focus or the system may simply automatically adjust the refractive power provided by the lenses 3, 4 to be zero.

At 41, the user provides input through the interface to confirm that they are looking at the second predetermined object and that they perceive it to be in focus. The measurement of the vergence exhibited by the user whilst looking at the second predetermined object is then measured at 32 to 39.

Since the vergence at the two set points has now been acquired, the microprocessor determines at 40 that it is not necessary to measure the vergence at any more set points and an equation relating the degree of vergence exhibited by the user to the focal length of the lenses 3, 4 is generated at 41. This is a simple linear equation, which can be determined from the vergences (i.e. as interpupillary distances) measured at the first and second set points of the focal length above. This is valid because there is a linear relationship between the vergence and the distance at which an object being viewed lies from the user. Thus, the relationship between vergence and the additional refractive power that must be provided for a user is also linear. In embodiments, where three or more set points are used, linear regression may be used to generate the equation.

This equation is used at 42 to populate a lookup table stored in memory 15. This can be done by using a range of values of interpupillary distance (as a measure of vergence) between the two values measured at each of the two set points as an input to the equation generated at 41. The result from the equation will be the associated focal length of the lenses 3, 4 at each of the range of values of interpupillary distance. Thus, corresponding pairs of values for the interpupillary distance and the focal length are obtained at a range of points between the two set points. Each value of interpupillary distance in the range is stored in the look-up table as an addressing or indexing variable to the look-up table in a linked relationship with a signal level for an actuation control signal required to adjust the lenses 3, 4 to the focal length corresponding to the interpupillary distance. In other embodiments, the equation itself may be stored in the memory 15 and used to calculate the focal length of lenses 3, 4 corresponding to a measured value of interpupillary distance each time it is required when in the operational mode.

In another embodiment, rather than allowing the user to adjust the focal length of lenses 3, 4, the focal length of the lenses can be set to prescribed refractive powers measured by an optician during an eye examination for both near and distance vision when the user is looking at nearby (e.g. reading a book) and distant objects (e.g. looking at a car in the distance) respectively. The user would then simply confirm using the interface that they are looking at the nearby or distant object, the parameters of the prescription (from which the required focal length of the lenses 3, 4 can be determined) having already been entered into the interface either by the user or by an eyecare professional.

FIG. 4 shows a flowchart for the method performed by the system (and in particular by microprocessor 14 when executing the program code stored in memory 15) shown in FIG. 2 in normal operation (i.e. when no longer in the calibration mode). Typically, the calibration method of FIG. 3 will already have been carried out. The part of the method starting at 50 and ending at 57 is identical to the part of the method of FIG. 3 starting at 32 and ending at 39. Since this has already been discussed above in detail, a description of this part of the method of FIG. 4 will not be repeated here.

The interpupillary distance measured at 57 is used to access the look-up table stored in memory 15 and populated during the calibration process of FIG. 3 at 42. The look-up table links the values of interpupillary distance to a corresponding signal level for an actuation control signal. This actuation control signal is then applied at 59 to the actuation system comprising stepper motor driver 18 and the miniature stepper motors 12 and 13. Stepper motor driver 18 monitors the current positions of the stepper motors 12 and 13 and converts the actuation control signal to an appropriate series of pulses to drive the stepper motors 12 and 13 to the required new position depending on the current interpupillary distance and the new position. Since stepper motors 12 and 13 are mechanically coupled to lenses 3 and 4 (as depicted by the dashed lines in FIG. 2), the focal length of the lenses 3 and 4 are adjusted to the appropriate value depending only on the degree of vergence exhibited by a user.

In a practical embodiment, steps 51 to 59 will be repeated in a cyclic loop. This is unlikely to be done continuously as it would cause the adjustment of the lenses to hunt all the while as the user's eyes exhibited different degrees of vergence. Instead, a time delay of a few seconds will be built in after each time the control signal is applied at 59 before a new image is acquired at 21.

1. A pair of spectacles comprising:
a pair of variable focal length lenses;
an image acquisition system adapted to acquire images of each of a user's eyes; and
a controller adapted to analyze the images to monitor the degree of vergence of the user's eyes, and to adjust a focal length of the variable focal length lenses to a value derived directly from the monitored degree of vergence.

2. The pair of spectacles according to claim 1, wherein the acquired images include corresponding parts of each of the user's eyes; and the controller is adapted to analyze the images to monitor the degree of vergence of the user's eyes by monitoring the distance between the corresponding parts.

3. The pair of spectacles according to claim 2, wherein the image acquisition system is adapted to acquire images including at least part of both of a user's pupils, and the controller is adapted to analyze the images to monitor the interpupillary distance.

4. The pair of spectacles according to claim 2, wherein the image acquisition system comprises one or more cameras for acquiring images including the corresponding parts of each of the user's eyes.

5. The pair of spectacles according to claim 2, wherein the image acquisition system comprises one or more light sources arranged to illuminate the user's eyes, thereby to form one or more Purkinje images in each of the user's eyes, corresponding Purkinje images in each of the user's eyes defining the corresponding parts of each of the user's eyes.

6. The pair of spectacles according to claim 5, wherein the controller is adapted to analyze the images to monitor the distance between the corresponding parts of each of the user's eyes by detecting the corresponding Purkinje images, calculating the location of the centroid of each of the corresponding Purkinje images, and calculating the distance between the centroids.

7. The pair of spectacles according claim 1, wherein the controller is further adapted to adjust the focal lengths by retrieving an actuation control signal level from a look-up table, and applying the actuation control signal level to one or more actuators coupled to the variable focal length lenses for adjusting their focal lengths.

8. The pair of spectacles according to claim 1, wherein the controller is further adapted to adjust the focal lengths by calculating an actuation control signal level from an equation relating the degree of vergence of the user's eyes to the focal length of the variable focal length lenses, and applying the actuation control signal level to one or more actuators coupled to the variable focal length lenses for adjusting their focal lengths.

9. The pair of spectacles according to claim 1, wherein the controller is located entirely on or within a frame housing the pair of variable focal length lenses.

10. The pair of spectacles according to claim 1, wherein the controller is switchable into a calibration mode, in which the controller is further adapted to acquire images of each of a user's eyes, adjust the focal length of the variable focal length lenses to each of at least two set points in succession, receive user input at each of the set points to allow a user to indicate when looking at a predetermined object, analyze the images acquired by the image acquisition system to monitor the degree of vergence of the user's eyes in response to receipt of the user input, and generate an equation relating the degree of vergence of the user's eyes to the focal length of the variable focal length lenses from the focal length and the monitored degree of vergence at each set point.

11. The pair of spectacles according to claim 10, wherein the controller is adapted to adjust the focal length of the variable focal length lenses to at least one of the set points in response to user input, whereby each set point represents the focal length at which the user perceives the predetermined object to be in focus.

12. The pair of spectacles according to claim 10, wherein the controller is adapted to adjust the focal length of the variable focal length lenses automatically to a set point associated with infinity focus.

13. The pair of spectacles according to claim 10, wherein the user input that allows the user to indicate when looking at a predetermined object additionally allows the user to indicate that the predetermined object is perceived to be in focus by the user.

14. The pair of spectacles according to claim 10, wherein there are only two set points.

15. The pair of spectacles according to claim 10, wherein the equation is a linear equation.

16. The pair of spectacles according to claim 10, wherein the controller is further adapted when in the calibration mode to use the equation to populate a look-up table linking the monitored degree of vergence or distance of the user's eyes to an actuation control signal level for causing one or more actuators to adjust the variable focal length lenses.

17. The pair of spectacles according to claim 10, wherein the controller is further adapted to store one or more parameters representing the equation.

18. A method of controlling the focal length of a pair of variable focal length lenses in a pair of spectacles, the method comprising:
acquiring images of each of a user's eyes;
analyzing the images to monitor the degree of vergence of the user's eyes; and
adjusting a focal length of the variable focal length lenses to a value derived directly from the monitored degree of vergence.

19. The method according to claim 18, wherein the acquired images include corresponding parts of each of a user's eyes; and the images are analyzed to monitor the degree of vergence of the user's eyes by monitoring the distance between the corresponding parts.

20. The method according to claim 19, further comprising acquiring images including at least part of both of a user's pupils, and analyzing the images to monitor the interpupillary distance.

21. The method according to claim 19, further comprising illuminating the user's eyes to form one or more Purkinje images in each of the user's eyes, corresponding Purkinje images in each of the user's eyes defining the corresponding parts of each of the user's eyes.

22. The method according to claim 21, further comprising analyzing the images to monitor the distance between the corresponding parts of each of the user's eyes by detecting the corresponding Purkinje images, calculating the location of the centroid of each of the corresponding Purkinje images, and calculating the distance between the centroids.

23. The method according to claim 18, further comprising
adjusting the focal lengths by retrieving an actuation control signal level from a look-up table, and
applying the actuation control signal level to one or more actuators coupled to the variable focal length lenses for adjusting their focal lengths.

24. The method according to any of claim 18, further comprising:
adjusting the focal lengths by calculating an actuation control signal level from an equation relating the degree of vergence of the user's eyes to the focal length of the variable focal length lenses, and
applying the actuation control signal level to one or more actuators coupled to the variable focal length lenses for adjusting their focal lengths.

25. The method of calibrating a pair of variable focal length lenses in a pair of spectacles according to claim 10, the method further comprising:
switching to the calibration mode;
acquiring images of each of a user's eyes;
adjusting the focal length of the variable focal length lenses to each of at least two set points in succession;
receiving user input at each of the set points to allow a user to indicate when looking at a predetermined object;
analyzing the images to monitor the degree of vergence of the user's eyes in response to receipt of the user input; and
generating an equation relating the degree of vergence of the user's eyes to the focal length of the variable focal length lenses from the focal length and the monitored degree of vergence at each set point.

26. The method according to claim 25, wherein the focal length of the variable focal length lenses is adjusted to at least one of the set points in response to user input, whereby each set point represents the focal length at which the user perceives the predetermined object to be in focus.

27. The method according to claim 25, wherein the focal length of the variable focal length lenses is adjusted automatically to a set point associated with infinity focus.

28. The method according to claim 25, wherein the user input that allows the user to indicate when looking at a predetermined object additionally allows the user to indicate that the predetermined object is perceived to be in focus by the user.

29. The method according to claim 25, wherein there are only two set points.

30. The method according to claim 25, wherein the equation is a linear equation.

31. The method according to claim 25, further comprising using the equation to populate a look-up table linking the monitored degree of vergence or distance exhibited by the user to an actuation control signal level for causing one or more actuators to adjust the variable focal length lenses.

32. The method according to claim 25, further comprising storing one or more parameters representing the equation.
